(19) United States
(12) Patent Application Publication
(10) Pub. No.: US 2005/0250188 A1
(43) Pub. Date: Nov. 10, 2005

(54) Vegetable protein fraction with phospholipase d activity

(76) Inventors: Renata Ulbrich-Hofmann, Halle (DE); Lydia Bezakova, Bratislava (SK); Marek Oblozinsky, Bratislava (SK)

Correspondence Address: FULBRIGHT & JAWORSKI, LLP, 666 FIFTH AVE, NEW YORK, NY, 10103-3198, US

(21) Appl. No.: 10/526,035

(22) PCT Filed: Aug. 29, 2003
(86) PCT No.: PCT/EP03/09625

§ 371 Date: Feb. 28, 2005

(30) Foreign Application Priority Data
Aug. 30, 2002 (DE) 10240154.3

(51) Int. Cl. C12P 13/00; C12N 9/20

(52) U.S. Cl. 435/128; 435/198

(57) ABSTRACT

What is claimed is a novel plant phospholipase D activity-containing protein fraction which is derived from representatives of the Papaveraceae family, and, in particular, from Papaver somniferum, and which is composed of two protein subfractions A and B whose molecular masses are, in particular, 116.4 kDa and, respectively, 114.1 kDa, which have isoelectric points, pI, of 8.7 and, respectively, 6.7 and which have hydrolytic activity optima at pH 8.0 and, respectively, 5.5. The two subfractions are activated by Zn²⁺ ions, in particular, and constitute two isoenzymes. This protein fraction is used, in particular, for hydrolyzing and/or transphosphatidylating phospholipids and their lyso forms.

The present invention relates to a plant protein fraction which possesses phospholipase D activity.

Phospholipase D (PLD), which is a phosphatidylcholine choline hydrolase (EC 3.1.4.4), is an important enzyme of phospholipid metabolism and is widespread in nature.

Phospholipase D is assigned to an enzyme class whose representatives are able, in heterogeneous systems, to transform water-insoluble substrates since they are able to catalyze reactions at the interface between a lipid and water. The fact that phospholipase D exhibits this amphiphilic behavior has resulted in this enzyme appearing to be of particular interest for science, such that a large number of different phospholipases D (species) could be isolated from a very wide variety of sources during the past decades. In particular, PLD is thought to be involved in cell-regulatory activities in connection with the intercellular signal exchange. PLD enzymes are also able, by way of their hydrolysis activity, to transfer phosphatidyl residues to alcohols. In a general manner, therefore, PLD variants are employed for the biocatalytic exchange of phospholipid head groups.

Thus, for example, enzyme fractions are known from sugarbeet, spinach or cabbage leaf plastids and from carrot chromoplasts. Corresponding fractions possessing phospholipase D activity have also been isolated from mitochondria and microsomes of immature peanut seeds as well as from castor oil seeds, Arabidopsis species and tomatoe.

Success has also been achieved in extracting a corresponding enzyme from defatted cottonseed meal.

In addition to plant sources, microorganisms also serve as a source, whereas, in particular, corynebacteria (Corynebacterium ovis), Escherichia coli, baker's yeast cells and streptomycetes (Streptomyces hachijoensis) are to be mentioned.

However, it has also been possible to isolate phospholipase from mammalian cells, for example from human eosinophils and rat brain microsomes.

The known phospholipase D enzymes present a heterogeneous picture in regard to their molecular weights:

Thus, the soluble enzyme isolated from cotton seed has a molecular weight of 71 000 3000 Da; phospholipase D from peanut seeds has a molecular weight of 200 000 10 000 Da and PLD from human eosinophils has a molecular weight of approx. 60 000 Da.

Corresponding bacterial enzymes, as can be isolated, for example, from Corynebacterium ovis, possess a molecular weight of approximately 90 000 Da.

With regard to the isoelectric point, phospholipase D from peanut seeds is known to have pI values of 4.65 while, on the other hand, the pI of a crude extract from human eosinophils is between 4.8 and 5.0 and, as a result of additional purification, can reach a value of between 5.8 and 6.2.

R. Lambrecht et al. (“A facile purification procedure of phospholipase D from cabbage and its characterization”; (1992) Biol. Chem. Hoppe Seyler Vol. 373 (2) 81-88) describe purification of PLD from white cabbage in two steps. This method comprises an ammonium sulfate precipitation and a subsequent Ca⁺-mediated affinity chromatography.

The publication by I. Schaffner et al. (“Genomic structure, cloning and expression of two phospholipase D isoenzymes from white cabbage”, Eur. J. Lipid Sci. Technol. 104, 79-87 (2002); corresponding to dissertation (2001)) describes how recombinant phospholipase D-active isoenzymes can be obtained from white cabbage using cloning methods and how these isoenzymes can be characterized in regard to their specific hydrolysis activity in dependence on the pH and on the Ca²⁺ concentration as well as in regard to their transphosphatidylating properties.

The review article by Michael Heller in Advanced Lipid Research, 1978, Volume 16, pages 267 to 326, provides a general overview of the state of knowledge with regard to phospholipase D.

In “Identification of two isoenzymes of phospholipase D from opium poppy” (Direct submission (2001) NCBI GenBank, accession Nos. AAL48261-AAL48264 and multiple sequence comparison), A. Lerchner et al. describe two truncated phospholipase D1 polypeptides, as well as two other truncated phospholipase D2 polypeptides, from Papaver somniferum. As can be seen from the multiple sequence comparison, the part amino acid sequences of proteins D1 and D2 exhibit a sequence identity of 98% to each other. In addition, the part sequences which are described possess a high degree of homology (70-84%) with the well-characterized phospholipase D varieties of the type. However, since the sequence determination is not complete at the 5′ end, it is not possible to assign the phospholipase D1 and D2 polypeptides to a defined enzyme.

The database entry by A. Lerchner et al. “Identification of two isoenzymes of phospholipase D from opium poppy” (Direct submission (2001) NCBI GenBank, accession Nos. AF451979-AF451982) describes nucleic acid sequences for the two polypeptides PLD1 and PLD2 which have just been mentioned.

Poppy seeds are known to be able to form secondary metabolites on an unusually large scale. Thus, alkaloids such as thebaine can, for example, be detected in poppy seeds after only a few days of swelling, thereby making the seeds of interest with regard to opium isolation, in particular.

Since phospholipase D (PLD) plays an evermore important role in the industrially employed catalytic hydrolysis of glycerophospholipids such as phosphatidylcholine (PC) to phosphatidic acid (PA), and also in transphosphatidylating processes in regard to headgroup exchange in phospholipids, the object of the present invention was that of isolating novel plant protein fractions which originate from representatives of the Papaveraceae family and which possess phospholipase D activity.

This object was achieved by means of a corresponding protein fraction which is characterized in that
a) it consists of two protein subfractions A and B, and
b) it can be activated by Zn²⁺ ions, and also
c) the subfractions A and/or B possess carbohydrate moieties,
   and with the protein subfraction A only possessing hydrolysis activity.

In accordance with the definition, the term “protein fraction” which is employed below encompasses all actual protein fractions and proteins as well as their possible variants, and also all enzymes and enzyme variants, all of which possess corresponding PLD activity.

Surprisingly, it was possible to establish that this plant protein fraction contains two isoenzyme units which both exhibit a relatively narrow molecular mass spectrum and whose activity optima lie in the strongly acidic region, on the one hand, but in the slightly basic region on the other hand. In addition, it was not to be expected, on the basis of the previously known PLDs, that isoenzymes from poppy possessing PLD activity can be activated with zinc, something which is advantageous particularly in regard to their use for preparing phospholipids, which are known to form mostly insoluble complexes with Ca ions. In addition, it was not possible to assume, on the basis of previously available findings with phospholipases D from plant sources, that protein fractions with corresponding activities will be found in representatives of the Papaveraceae family.

The present invention claims, in particular, a protein fraction which is derived from Papaver somniferum (opium poppy) and very particularly preferably from developing seedlings and/or endosperms. It is naturally also possible to conceive of a variant in which the protein fractions are derived indirectly from Papaver in that the protein fractions are obtained namely by means of recombinant methods, in particular using recombinant microorganisms which contain the genes for the corresponding protein fraction.

As already explained, one essential aspect according to the invention is directed towards the fact that the claimed plant protein fraction contains two isoenzymes. In this regard, the present invention prefers a protein fraction whose subfraction A possesses a molecular mass between 116 and 118 kDa, an isoelectric point pI between 8.5 and 8.9 and a hydrolytic activity optimum at pH values between 7.8 and 8.2, and the subfraction B subfraction B possesses a molecular mass between 112 and 115 kDa, an isoelectric point pI between 6.5 and 6.9 and a hydrolytic activity optimum at pH values between 5.0 and 6.0.

A defined value for the isoelectric point can be obtained by subjecting isolated fractions to further purification.

For this reason, the protein fraction of the present invention is also, in particular, characterized by the fact that the subfraction A has an isoelectric point pI of 8.7 and a molecular mass of 116.4 kDa as well as a hydrolytic activity optimum at pH 8.0. The corresponding preferred values for subfraction B are 114.1 kDa with regard to the molecular mass and 6.7 with regard to the isoelectric point pI, with the hydrolytic activity optimum being at pH 5.5. These features are also encompassed by the present invention.

As already mentioned, protein fractions possessing phospholipase D activity are usually calcium ion-dependent. However, this pronounced dependence has not proved to be true in the case of the claimed plant protein fraction from Papaveraceae, which is imperatively Zn²⁺ ion-activatable. However, the activity optimum of this protein fraction can also be reached in the presence of calcium ion concentrations, which are then usually between 40 M and 100 mM, with corresponding enzyme activities appearing at concentrations of between 2 and 20 mM and between 5 and 15 mM.

With regard to subfraction B, the present invention claims a protein variant whose activatability optimum occurs in the presence of Zn²⁺ ion concentrations which are between 1.0 and 10 mM and, particularly preferably, at 5 mM.

As what is essential for the invention, inter alia, the present invention provides for subfractions A and/or B to possess carbohydrate moieties such that they are consequently present in glycosylated form as N-linked glycoproteins, and for subfractions A and B to be isoenzymes.

In conformity with the surprisingly different activity properties of subfractions A (only hydrolysis) and B (pronounced transphosphatidylation), the present invention also encompasses a variant of the protein fraction in which the transphosphatidylation activity is, all in all, more strongly expressed than its hydrolysis activity, something which can be explained, in particular, by the individual activities of the subfractions and is also of importance with regard to the previously known PLD variants, as compared with which the novel protein fractions exhibit transphosphatidylation activities which are up to 100 times more pronounced, based on the corresponding hydrolysis activities.

In addition to the protein fraction itself, the present invention also claims the use of this protein fraction for hydrolyzing and/or transphosphatidylating phospholipids and/or their lyso forms, with, in particular, the synthesis of phosphatidylcholine, phosphatidylethanolamine, phosphatidylglycerol, phosphatidylinositol, phosphatidic acid and phosphatidylserine, and their lyso forms and any arbitrary mixtures, being claimed. In this connection, it is also worth mentioning the fact that the protein fraction according to the invention is able to hydrolyze phosphatidylinositol and/or to carry out a headgroup exchange on PI, something which the previously known PLDs are likewise not known to do. While, in this connection, the conduct of the reaction as a whole is not to be regarded as being critical, organic and/or aqueous phases have proved to be very suitable as reaction media while phosphatidylcholine and phosphatidylethanolamine have proved to be very suitable as the phospholipid source.

In summary, it can be stated that, by means of this novel phospholipase D activity-possessing plant protein fraction obtained from representatives of the Papaveraceae family, success has been achieved in isolating a PLD which, taken overall, possesses a very pronounced transphosphatidylation activity and whose two isoenzymic subfractions exhibit hydrolytic activities, in particular towards phosphatidylcholine; while both subfractions can be activated by calcium ions, as is a known property of PLDs, the novel protein fraction can, in contrast to known phospholipase D variants from other plants, also, and in particular, be activated by Zn²⁺ ions.

The novel protein fraction consequently differs fundamentally from the previously known plant PLDs and its properties differ markedly from those of the PLDs whose gene sequences have already been determined.

The following examples clarify the characteristic features of the claimed plant protein fraction possessing phospholipase D activity.

### EXAMPLES

Working-Up of Plant Material (Enzyme Isolation)

Poppy seeds (Papaver somniferum), which were present on a 10 mm thick polyurethane foam layer in Petri dishes which were covered with a nylon fabric, were germinated in distilled water. The germination process was carried out in the dark, at 25° C. and at from 70 to 80% relative humidity. On the second day after the swelling, the endosperm was removed from the seedlings.

These endosperms, which were obtained from a total of 10 g of fresh poppy seeds, were triturated with a small quantity of cold acetone in a mortar and then homogenized with 300 ml of cold acetone which contained 300 g of solid CO₂. The resulting precipitate was then washed with cold acetone until the filtrate was colorless and transparent. In powder form, the vacuum-dried residue was stable for several months at 4° C.

2 g of this acetone powder were homogenized in 50 ml of a mixture consisting of 0.1 molar sodium acetate buffer/10 mM CaCl₂/6 mM cysteine hydrochloride (pH 5.5) and centrifuged at 12 000 g and 4° C. for 10 minutes. The resulting extracts were treated with (NH₄)₂SO₄ (60% saturation) and centrifuged at 26 000 g and 4° C. for 45 minutes. The precipitate was then taken up in a preferably minor quantity of a mixture, consisting of 0.01 sodium acetate buffer/10 mM CaCl₂/6 mM cysteine hydrochloride (pH 5.5). After dialysis against a mixture composed of 0.01 molar sodium acetate buffer/50 mM CaCl₂ (pH 5.5), the enzyme solution was loaded onto an octyl-Sepharose CL-4B column. The proteins were eluted at a flow rate of 9 ml/h in three steps using the following solutions: 0.01M sodium acetate buffer/50 mM CaCl₂; 0.005 M sodium acetate buffer/30 mM CaCl₂ (pH 5.5). 0.005 M sodium acetate buffer/0.1 mM ethylenediaminetetraacetic acid (EDTA), pH 5.5. The enzyme activity was determined at pH values of 5.5 and 8.0 using phosphatidyl-p-nitrophenol (PpNP), and the combined active fractions were concentrated using a 100 kDa membrane.

Protein Determination

The protein content was determined in accordance with the standard method of M. M. Bradford (Anal. Biochem. 72, 1976, 248-254) using bovine serum albumin as standard.

Hydrolytic Activity in Aqueous Systems

The hydrolytic activity of the resulting plant protein fraction possessing phospholipase D activity was determined in an aqueous system by determining the p-nitrophenol which was released from PpNP (Method in accordance with P. D'Arrigo, V. Piergianni, D. Scarcelli, S. Servi, “A Spectrophotometric Essay for Phospholipase D”, Anal. Chim. Acta, 304, 1995, 249-254).

In order to characterize the respective activities of the PLD subfractions A and B, the reactions were carried out at different pH values in the presence of 10 mM CaCl₂, in the presence of different concentrations of CaCl₂ at pH values of 5.5 and, respectively, 8.0, and also in the presence of different concentrations of ZnCl₂ at a pH of 5.5.

Transphosphatidylation and Hydrolytic Activity in a Two-Phase System

In conformity with the method according to N. Dittrich and R. Ulbrich-Hofmann (“Transphosphatidylation by immobilised Phospholipase D in aqueous media”, Biotechnol. Appl. Biochem. 34, 2001, 189-194), the transphosphatidylation and hydrolysis activities were determined in a two-phase system. The corresponding reaction media were composed of a phosphatidylcholine-containing diethyl ether, glycerol (for determining the transphosphatidylation activity) or water (for determining the hydrolysis activity) as well as a mixture composed of Tris-HCl/CaCl₂ or a mixture composed of sodium acetate buffer/CaCl₂ and a purified enzyme (PLD-A and PLD-B). The respective reactions were carried out at 30° C., and at a shaking frequency of 250/min, in reaction vessels which were sealed with Teflon-silicone septa. During the reaction, aliquots of the organic phase(s) were analyzed by means of HPTLC. The phospholipid contents were determined densitometrically at 550 nm in comparison with standard mixtures composed of phosphatidylcholine, phosphatidic acid and phosphatidylglycerol, with the enzyme activity being calculated from the increase in phosphatidyl-glycerol or phosphatidic acid.

Determining Enzyme Features

The molecular mass of the purified proteins was determined by means of electrophoresis in the presence of SDS using a Bio-Rad Mini Protein II gel electrophoresis cell and polyacrylamide gels.

The isoelectric points of the PLD subfractions A and B were determined using a PhastGel IEF 3-9 on a FastSystem separation and control unit (Pharmacia. LKB Biotechnology), with pI IEF markers (liquid mix 3-10) being employed for the calibration. The proteins were stained with Coomassie Brilliant Blue G-250.

For the glycoprotein determination, an SDS-PAGE gel which contained PLD-A, PLD-B and peroxidase (as standard for a glycosylated protein) and also aldolase (as standard for a nonglycosylated protein) was brought into contact, at 300 V and 5 mA/cm², with a nitrocellulose membrane for a period of 180 minutes. After the proteins had transferred, the total carbohydrate content was determined using an ECL glycoprotein detection module.

The N-terminal amino acids were sequenced using a 492 cLC protein sequencer (PE Applied Biosystems).

Results:

The two enzyme forms which were obtained by means of hydrophobic interaction chromatography for purifiying PLD, in accordance with R. Lambrecht and R. Ulbrich-Hofmann (“A facile purification procedure of phospholipase D from cabbage and its characterization”, Biol. Chem. Hoppe-Seyler 373, 1992, 81-88), were active at pH 8.0 (PLD-A) and, respectively, pH 5.5 (PLD-B). The same purification results could be achieved by replacing CaCl₂ with ZnCl₂ in buffer solutions. Both enzyme subfractions were homogeneous in an SDS-PAGE gel. Table 1 shows the purification data, with the purification factors for the two isoenzymes being 84.7 (PLD-A) and, respectively, 94.1 (PLD-B).

Protein Determination of the Two Subfractions

Using the SDS-PAGE method, the molecular masses of PLD subfraction A and PLD subfraction B were determined to be 116.4 and, respectively, 114.1 kDa. Their isoelectric points were 8.7 (PLD-A) and 6.7 (PLD-B). It was demonstrated that both PLD-A and PLD-B were present in glycosylated form since a positive deglycosylation reaction using N-glycosidase F showed the presence of an N-bound carbohydrate in the case of both subfractions. Since the N-terminal sequencing method failed in the case of both subfractions, it is to be assumed that an N-terminal modification is present in both cases.

pH Activity Profiles

Significant differences were found in the hydrolytic activities of PLD subfractions A and B toward PpNP and as a function of the pH. Subfraction A possesses a sharp pH optimum at pH 8.0 while subfraction B has no particularly marked activity at this pH. By contrast, the pH optimum of subfraction B is at pH 5.5, at which subfraction A scarcely exhibits any activity. Under conditions which are in each case optimal, subtraction B exhibits an activity which is 38% higher than that of subfraction A.

Influence of Metal Ions

In regard to the fact that PLD variants are known to be activated by Ca²⁺ ions, the novel subfractions A and B from opium poppy were also found to exhibit calcium ion dependency, with an activity maximum being obtained at a CaCl₂ concentration of 10 mM. It was only possible to obtain very slight activation of the two subunits with Mg²⁺ ions, whereas Zn²⁺ ions activated the PLD subtractions A and, in particular, B more strongly than did calcium ions. Subfraction B was activated four times more strongly by an optimal Zn²⁺ ion concentration (5 mM) than it was by the optimal Ca²⁺ ion concentrations.

Transphosphatidylating Activities and Hydrolytic Activities in a 2-Phase System

The transphosphatidylating potentials of subfractions A and B were determined, by means of HPTLC and densitometric quantification of the reaction products, in a biphasic system which was composed of a sodium acetate buffer (pH 5.5) or Tris-HCl buffer (pH 8.0) and 40 mM CaCl₂, diethyl ether which contained phbsphatidylcholine as substrate and glycerol as acceptor alcohol. At pH 5.5, PLD subtraction B possessed high transphosphatidylating potential since more than 80% of the phosphatidylcholine had been converted into the transphosphatidylation product phosphatidylglycerol after 240 minutes, whereas it was not possible to find any phosphatidic acid at all under these reaction conditions. PLD-B did not exhibit any transphosphatidylation or hydrolysis at a pH of 8.0. PLD subtraction A did not exhibit any transphosphatidylating activity either at pH 5.5 or pH 8.0; as expected, however, it exhibited pronounced hydrolysis activity at pH 8.0

TABLE 1
Purification of protein fractions with PLD activity from opium
poppy seeds. The hydrolytic PLD activity of the protein fraction
towards PpNP was determined at pH 8.8 and 5.0.

| | | | | Specific activity | |
|---|---|---|---|---|---|
| | | Activity | | [μmol min⁻¹ | |
| Purification | Protein | [μmol min⁻¹] | | mg⁻¹] | |
| step | [mg] | pH 8.0 | pH 5.5 | pH 8.0 | pH 5.5 |
| Crude extract* | 43.25 | 3.74 | 4.65 | 0.09 | 0.11 |
| (NH₄)₂SO₄ | 9.56 | 1.99 | 2.38 | 0.21 | 0.25 |
| precipitate** | | | | | |
| Octyl-Sepharose CL-4B | | | | | |
| Subfraction A (PLD-A) | 0.14 | 1.05 | — | 7.32 | — |
| Subfraction B (PLD-B) | 0.18 | — | 1.85 | — | 10.13 |

*after homogenizing the acetone powder and centrifuging at 12 000 g.
**after centrifuging the precipitate at 26 000 g and then dialyzing.

1. A plant protein fraction which is derived from representatives of the Papaveraceae family and which possesses phospholipase D activity, comprising
a) two protein subfractions A and B;
b) it can be activated by Zn²⁺ ions, and also
c) at least one of subfractions A or B possess carbohydrate wherein only protein subfraction A possesses hydrolysis activity.

2. The protein fraction as claimed in claim 1, derived from Papaver somniferum and very particularly preferably from developing seedlings or endosperms.

3. The protein fraction as claimed in claim 1 wherein the subfraction A possesses a molecular mass of between 116 and 118 kDa, an isoelectric point, pI, of between 8.5 and 8.9 and a hydrolytic activity optimum at pH values of between 7.8 and 8.2, and the subfraction B possesses a molecular mass of between 112 and 115 kDa, an isoelectric point, pI, of between 6.5 and 6.9 and a hydrolytic activity optimum at pH values of between 5.0 and 6.0.

4. The protein fraction as claimed in claim 1 wherein the subfraction A has a molecular mass of 116.4 kDa, an isoelectric point, pI, of 8.7 and a hydrolytic activity optimum at pH 8.0.

5. The protein fraction as claimed in claim 1, wherein the subfraction B has a molecular mass of 114.1 kDa, an isoelectric point, pI, of 6.7 and a hydrolytic activity optimum at pH 5.5.

6. The protein fraction as claimed in claim 1, wherein the subfraction B possesses an activatability optimum at Zn²⁺ ion concentrations of between 1.0 and 10 mM and, particularly preferably, at 5 mM.

7. The protein fraction as claimed in claim 1 wherein the subfractions A and B are isoenzymes.

8. The protein fraction as claimed in claim 1 wherein its transphosphatidylating activity of the protein fraction is more strongly pronounced than its hydrolysis activity.

9. A method comprising hydrolyzing or transphosphatidylating phospholipids or their lyso forms with the protein fraction of claim 1.

10. The method as claimed in claim 9 for synthesizing phosphatidylcholine, phosphatidylethanolamine, phosphatidylglycerol, phosphatidylinositol, phosphatidic acid and phosphatidylserine and their lyso forms.

11. The method as claimed in claim 9 phosphatidylinositol is hydrolized or a headgroup exchange is performed on phosphatidylinositol.
